(19) United States
(12) Patent Application Publication
(10) Pub. No.: US 2026/0159871 A1
(43) Pub. Date: Jun. 11, 2026

(54) ENZYME, ENZYME ELECTRODE, BIOSENSOR, BIOREACTOR, AND BIOFUEL CELL

(71) Applicants: Murata Manufacturing Co., Ltd., Nagaokakyo-shi, (JP); Kyoto University, Kyoto-shi (JP)

(72) Inventors: Hideyuki KUMITA, Nagaokakyo-shi (JP); Keisei SOWA, Kyoto-shi (JP); Taiki MAKIZUKA, Kyoto-shi (JP); Osamu SHIRAI, Kyoto-shi (JP); Yuki KITAZUMI, Kyoto-shi (JP); Yasuyoshi SAKAI, Kyoto-shi (JP); Hiroya YURIMOTO, Kyoto-shi (JP); Shinichi OIDE, Nagaokakyo-shi (JP); Masaki YAMAMOTO, Nagaokakyo-shi (JP)

(21) Appl. No.: 19/369,562

(22) Filed: Oct. 27, 2025

Related U.S. Application Data
(63) Continuation of application No. PCT/JP2024/003218, filed on Feb. 1, 2024.

(30) Foreign Application Priority Data
Apr. 27, 2023 (JP) 2023-073537
Oct. 24, 2023 (JP) 2023-182625

(51) Int. Cl. C12Q 1/32; C12M 1/42; C12Q 1/00

(52) U.S. Cl. CPC C12Q 1/32; C12M 35/08; C12Q 1/005; G01N 2333/9029

(57) ABSTRACT

An enzyme electrode that includes: an electrode base material; a first catalyst layer on the electrode base material, the first catalyst layer including an enzyme (a1) capable of catalyzing an oxidation-reduction reaction of a redox couple of NAD(P)H and NAD(P) and capable of direct electron exchange between the enzyme (a1) and the electrode base material accompanying the oxidation-reduction reaction; and a second catalyst layer on the first catalyst layer, the second catalyst layer including an NAD(P)H or NAD(P) dependent oxidoreductase (b) and NAD(P)H and/or NAD(P).

### CROSS REFERENCE TO RELATED APPLICATIONS

The present application is a continuation of International application No. PCT/JP2024/003218, filed Feb. 1, 2024, which claims priority to Japanese Patent Application No. 2023-073537, filed Apr. 27, 2023, and Japanese Patent Application No. 2023-182625, filed Oct. 24, 2023, the entire contents of each of which are incorporated herein by reference.

### REFERENCE TO AN ELECTRONIC SEQUENCE LISTING

The sequence listing of the present application is submitted electronically as an XML file named “040857-00093_Sequence_Listing.xml”, created on Jan. 26, 2026, and having a size of 4,367 bytes. This sequence listing submitted electronically is an integral part of the specification and is incorporated herein by reference in its entirety.

### STATEMENT REGARDING PRIOR DISCLOSURE BY THE INVENTOR OR A JOINT INVENTOR

The subject matter claimed in this application was disclosed by at least one of the inventors in an article titled “Multiple electron transfer pathways of tungsten-containing formate dehydrogenase in direct electron transfer-type bioelectrocatalysis” (Chemical Communications, 2022, Vol. 58, pp. 6478-6481), published on Apr. 29, 2022. This disclosure is excepted from prior art under 35 U.S.C. 102(b)(1)(A).

The subject matter claimed in this application was disclosed by at least one of the listed inventors in an article on the Kyoto University website (URL:chrome-extension://efaidnbmnnnibpcajpcglclefindmkaj/https://www.kyoto-u.ac.jp/sites/default/files/2022-05/220520_sowa-924dfdec67fa6b7f7ae1775d53065834.pdf) titled “Elucidating the Electron Transfer Mechanism of CO₂-Converting Enzymes” and was published on May 20, 2022. This disclosure is excepted from prior art under 35 U.S.C. 102(b)(1)(A).

The subject matter claimed in this application was disclosed by at least one of the listed inventors in an article on the Osaka University website (URL: https://resou.osaka-u.ac.jp/ja/research/2022/20220520_2) titled “Elucidating the Electron Transfer Mechanism of CO₂-Converting Enzymes” and was published on May 20, 2022. This disclosure is excepted from prior art under 35 U.S.C. 102(b)(1)(A).

The subject matter claimed in this application was disclosed by at least one of the listed inventors in the abstract book of the 68th Annual Meeting of the Polarographic Society of Japan (p. 58), titled “Biological electrochemical study on direct electron transfer type NAD regeneration system and NAD-dependent enzymes” and published on Oct. 24, 2022. This disclosure is excepted from prior art under 35 U.S.C. 102(b)(1)(A).

The subject matter claimed in this application was disclosed by at least one of the listed inventors during a presentation titled “Biological electrochemical study on direct electron transfer type NAD regeneration system and NAD-dependent enzymes” at the “68th Annual Meeting of the Polarographic Society of Japan” held at the Katsura Campus of Kyoto University on Nov. 11, 2022. This disclosure is excepted from prior art under 35 U.S.C. 102(b)(1)(A).

The subject matter claimed in this application was disclosed by at least one of the listed inventors in the abstract book of the 3rd Kansai Electrochemistry Meeting Webinar (p. 3), titled “Acceleration of direct electron transfer type NAD regeneration system by enzymatic engineering approach” and published on Dec. 5, 2022. This disclosure is excepted from prior art under 35 U.S.C. 102(b)(1)(A).

The subject matter claimed in this application was disclosed by at least one of the listed inventors during a presentation titled “Acceleration of direct electron transfer type NAD regeneration system by enzymatic engineering approach” at the 3rd Kansai Electrochemistry Meeting Webinar on Dec. 10, 2022. This disclosure is excepted from prior art under 35 U.S.C. 102(b)(1)(A).

The subject matter claimed in this application was disclosed in the Master's Thesis for Academic Year 2022, titled “Consideration of electron transfer pathway and its application in single expression of formate dehydrogenase diaphorase subunit,” by the inventor, Taiki Makizuka, and which was published on Feb. 2, 2023. This disclosure is excepted from prior art under 35 U.S.C. 102(b)(1)(A).

The subject matter claimed in this application was disclosed by one of the inventors, Taiki Makizuka, in a poster presentation titled “Consideration of electron transfer pathway and its application in single expression of formate dehydrogenase diaphorase subunit” at the “Master's Program Poster Session of the Division of Applied Life Sciences,” held at Kyoto University on Feb. 3, 2023. This disclosure is excepted from prior art under 35 U.S.C. 102(b)(1)(A).

The subject matter claimed in this application was disclosed by one of the inventors, Taiki Makizuka, during an oral presentation titled “Analysis of Direct Electron Transfer-type NAD⁺/NADH Regeneration System of Diaphorase Subunit and Its Application” at the “Master's Thesis Presentation Meeting of the Graduate School of Agriculture, Kyoto University,” held at Kyoto University, on Feb. 22, 2023. This disclosure is excepted from prior art under 35 U.S.C. 102(b)(1)(A).

The subject matter claimed in this application was disclosed by at least one of the listed inventors in the abstract book of the 90th Annual Meeting of the Electrochemical Society of Japan (Abstract No. 3E04) in an article titled “Construction of direct electron transfer type NAD⁺/NADH interconversion system for biotechnological applications” and which was published on Mar. 17, 2023. This disclosure is excepted from prior art under 35 U.S.C. 102(b)(1)(A).

The subject matter claimed in this application was disclosed by at least one of the listed inventors in a presentation titled “Construction of direct electron transfer type NAD⁺/NADH interconversion system for biotechnological applications” at the “90th Annual Meeting of the Electrochemical Society of Japan” (The Electrochemical Society of Japan), on Mar. 29, 2023. This disclosure is excepted from prior art under 35 U.S.C. 102(b)(1)(A).

### TECHNICAL FIELD

The present disclosure relates to an enzyme, an enzyme electrode, a biosensor, a bioreactor, and a biofuel cell.

### BACKGROUND ART

As interest is recently growing in energy problems, biofuel cells, which use biological substances such as saccharides and alcohols as fuels, are attracting attention. A biofuel cell can generate power by using an enzyme as an electrocatalyst and combining an oxidation reaction of a fuel at an anode and a reduction reaction of oxygen or the like at a cathode.

In an anode of a biofuel cell, electrons are extracted from a fuel by the catalytic function of an enzyme and transmitted to the electrode. Enzymes widely used for performing an oxidation reaction of a fuel at an anode include a nicotinamide adenine dinucleotide (NAD) dependent oxidase and a nicotinamide adenine dinucleotide phosphate (NADP) dependent oxidase that function, respectively, using free nicotinamide adenine dinucleotide (NAD) and nicotinamide adenine dinucleotide phosphate (NADP) as a coenzyme. However, reactions using these catalysts generate NADH and NADPH, which have a very high overvoltage for direct electrolysis, and thus necessitate use of catalysts such as oxidation-reduction dyes and o-quinones.

In order to solve the above problem, a method has been proposed in which a substance called an electron mediator is used as a substance that catalyzes electron transfer between an enzyme and an electrode. An electrode reaction using an electron mediator as described above is called a mediator electron transfer (MET) type enzyme functional electrode reaction.

For example, Patent Literature 1 to Patent Literature 5 disclose biofuel cells obtained by combining an NAD(P)H or NAD(P) dependent oxidoreductase and a MET-type enzyme functional electrode reaction.
Patent Literature 1: JP 2004-71559 A
Patent Literature 2: JP 2012-151130 A
Patent Literature 3: JP 2012-178335 A
Patent Literature 4: JP 2009-69085 A
Patent Literature 5: JP 2018-68287 A

### SUMMARY OF THE DISCLOSURE

As described above, a MET-type biofuel cell has a problem that the electron mediator is to be immobilized so as not to dissipate from the enzyme-immobilized electrode.

In contrast, an electrode reaction called a direct electron transfer (DET) type enzyme electrode reaction has attracted attention as an ideal reaction system because in the reaction, an enzyme can directly transfer electrons to an electrode to eliminate the necessity of an electron transfer mediator that mediates electron transfer between the enzyme and the electrode and thus immobilization of the electron mediator becomes unnecessary.

The present disclosure has been made in view of the above circumstance, and an object of the present disclosure is to provide an enzyme electrode capable of direct electron exchange, between an electrode base material and an enzyme, accompanying an oxidation-reduction reaction.

The present inventors have extensively studied in order to solve the above problem, and have found that direct electron exchange accompanying an oxidation-reduction reaction is performed between an electrode base material and an enzyme in an electrode in which a first catalyst layer and a second catalyst layer are layered in this order on an electrode base material, the first catalyst layer includes an enzyme capable of catalyzing an oxidation-reduction reaction of a redox couple of NAD(P)H and NAD(P) and capable of direct electron exchange, between the enzyme and the electrode base material, accompanying the oxidation-reduction reaction, and the second catalyst layer includes an NAD(P)H or NAD(P) dependent oxidoreductase (b) and NAD(P)H and/or NAD(P), and thus the present inventors have conceived an excellent solution of the above problem and have arrived at the present disclosure.

The present disclosure is an enzyme electrode including: an electrode base material; a first catalyst layer on the electrode base material, the first catalyst layer including an enzyme (a1) capable of catalyzing an oxidation-reduction reaction of a redox couple of NAD(P)H and NAD(P) and capable of direct electron exchange between the enzyme (a1) and the electrode base material accompanying the oxidation-reduction reaction; and a second catalyst layer on the first catalyst layer, the second catalyst layer including an NAD(P)H or NAD(P) dependent oxidoreductase (b) and NAD(P)H and/or NAD(P).

The present disclosure is also an enzyme capable of catalyzing an oxidation-reduction reaction of a redox couple of NAD(P)H and NAD(P), the enzyme includes one flavin mononucleotide and two Fe—S clusters, the one flavin mononucleotide and at least one of the two Fe—S clusters have a distance of 2 nm or less, and at least one of the two Fe—S clusters is present at a distance of 2 nm or less from a surface of the enzyme.

The enzyme electrode of the present disclosure has the above-described configuration and is capable of direct electron exchange, between the electrode base material and the enzyme, accompanying an oxidation-reduction reaction, and thus can be suitably used in, for example, a biofuel cell, a biosensor, or a bioreactor.

### BRIEF DESCRIPTION OF THE DRAWINGS

FIG. 1 is a schematic diagram of an embodiment of an enzyme electrode of the present disclosure.

FIG. 2 is a diagram showing distances between cofactors in rFoDH1 obtained in Preparation Example 1.

FIG. 3 is a diagram showing distances from each cofactor to an enzyme surface in rFoDH1 obtained in Preparation Example 1.

FIG. 4 is a diagram showing distances from each cofactor to an enzyme surface in rFoDH1β (Me) obtained in Preparation Example 2.

FIG. 5 is a graph showing voltammograms in cyclic voltammetry (CV) measurement using enzyme electrodes 1 and 2 obtained in Preparation Examples 4 and 5.

FIG. 6 is a graph showing voltammograms in CV measurement performed using a measurement solution containing NAD-dependent glucose dehydrogenase and glucose with the enzyme electrode 2 obtained in Preparation Example 5.

FIG. 7 is a graph showing voltammograms in CV measurement performed using a measurement solution containing NAD-dependent glycerol dehydrogenase and dihydroxyacetone with the enzyme electrode 2 obtained in Preparation Example 5.

FIG. 8 is a graph showing voltammograms in CV measurement with an enzyme electrode 3 obtained in Preparation Example 6.

FIG. 9 is a graph showing voltammograms in CV measurement with an enzyme electrode 4 obtained in Preparation Example 7.

FIG. 10 is a graph showing a result of chronoamperometry (CA) measurement with an enzyme electrode 5 obtained in Preparation Example 8.

FIG. 11 is a graph showing voltammograms in CV measurement with enzyme electrodes 6 and 7 obtained in Preparation Examples 9 and 10.

### DESCRIPTION OF THE PREFERRED EMBODIMENTS

Hereinafter, the enzyme electrode, the enzyme, the biosensor, the bioreactor, and the biofuel cell of the present disclosure will be described.

However, the present disclosure is not limited to the configurations described below, and can be appropriately modified and applied without changing the gist of the present disclosure. The present disclosure also includes a combination of two or more preferable configurations of the present disclosure described below.

### [Enzyme Electrode]

The enzyme electrode of the present disclosure includes an electrode base material and a first catalyst layer that includes an enzyme (a1) capable of catalyzing an oxidation-reduction reaction of a redox couple of an oxidized form and a reduced form of nicotinamide adenine dinucleotide and/or nicotinamide adenine dinucleotide phosphate and capable of direct electron exchange, between the enzyme (a1) and the electrode base material, accompanying the oxidation-reduction reaction (hereinafter, also referred to as enzyme (a1)).

In the present description, an oxidized form and a reduced form of nicotinamide adenine dinucleotide are also described as NAD and NADH, respectively, and an oxidized form and a reduced form of nicotinamide adenine dinucleotide phosphate are also described as NADP and NADPH, respectively.

The term NAD(P)H means NADH or NADPH, and the term NAD(P) means NAD or NADP.

The enzyme electrode of the present disclosure further includes a second catalyst layer including an NAD(P)H or NAD(P) dependent oxidoreductase (b) (hereinafter, also referred to as enzyme (b)) and NAD(P)H and/or NAD(P), and the first catalyst layer and the second catalyst layer are layered in this order on the electrode base material.

The enzyme electrode of the present disclosure is suitable for an anode of a biofuel cell. NAD(P) receives electrons generated by oxidation of a substrate catalyzed by the oxidoreductase (b) and becomes NAD(P)H, the NAD(P)H is oxidized by the enzyme (a1) to generate electrons, and the electrons can be directly transferred to the electrode base material by the enzyme (a1).

The enzyme electrode of the present disclosure, as an anode of a biofuel cell, does not need an electron mediator in electron transfer between the electrode base material and the enzyme (a1).

The enzyme electrode of the present disclosure may include an electron mediator or no electron mediator, and preferably includes no electron mediator. The content of the electron mediator is preferably 200 mol % or less with respect to 100 mol % of the enzyme (a1). The content is more preferably 50 mol % or less, still more preferably 10 mol % or less, particularly preferably 1 mol % or less, most preferably 0 mol %.

The term electron mediator means a substance capable of exchanging electrons with an enzyme or a coenzyme and further exchanging electrons with a conductive base material. However, in the present description, NAD(P)H and NAD(P) are not an electron mediator.

The electron mediator is not limited, and examples of the electron mediator include metal complexes having a central metal that is a metal element such as Os, Fe, Ru, Co, Cu, Ni, V, Mo, Cr, Mn, Pt, or W or an ion of such a metal (for example, ferrocene, alkali metals ferricyanide such as potassium ferricyanide, lithium ferricyanide, and sodium ferricyanide, alkyl substituted products thereof (such as methyl substituted products, ethyl substituted products, and propyl substituted products), and potassium octacyanotungstate); quinones such as quinone, benzoquinone, anthraquinone, and naphthoquinone; heterocyclic compounds such as viologen, methylviologen, benzylviologen, phenazine methosulfate, phenazine ethosulfate, bipyridine, and derivatives thereof, and others including 2,6-dichlorophenolindophenol, methylene blue, β-naphthoquinone-4-sulfonic acid potassium salt, and vitamin K.

The enzyme electrode of the present disclosure is characterized in that the first catalyst layer and the second catalyst layer are layered in this order on the electrode base material.

The first catalyst layer is to include the enzyme (a1), and may include a solvent and a buffer component contained in a buffer.

Examples of the solvent include aqueous solvents such as water and ethanol. The solvent is preferably water.

Examples of the buffer component include phosphates such as potassium phosphate and sodium phosphate, imidazole, carbonates, borates, tartrates, citrates, tris(hydroxymethyl)aminomethane (TRIS), 4-(2-hydroxyethyl)-piperazine-1-ethanesulfonic acid (HEPES), and 3-morpholinopropanesulfonic acid (MOPS).

The second catalyst layer is to include the enzyme (b) and NAD(P)H and/or NAD(P), and may include a solvent and a buffer component contained in a buffer.

Examples of the solvent and the buffer component include those described for the first catalyst layer.

The enzyme electrode of the present disclosure does not limit a method of layering the first catalyst layer and the second catalyst layer on the electrode base material.

Examples of the method of layering the first catalyst layer and the second catalyst layer on the electrode base material include a method in which a composition containing the enzyme (a1) (hereinafter, also referred to as enzyme (a1)-containing composition) is applied to the electrode base material and dried. The application method may be any commonly used method, and examples of the method include a spin coating method, a spray method, a screen method, a dip coating method, and a blade method.

The enzyme (a1)-containing composition is to contain the enzyme (a1), and may contain a solvent and a buffer component. The enzyme (a1)-containing composition may contain an immobilizing agent such as a polymer or a crosslinking agent, and the enzyme (a1) may be more strongly immobilized to the electrode base material by the immobilizing agent.

The polymer is not limited, and examples of the polymer include polyvinylimidazole (PVI), polyallylamine, polyamino acids (such as polylysine), polypyrrole, polyacrylic acid, polyvinyl alcohol, a graft copolymer of polypropylene and maleic anhydride, a copolymer of methyl vinyl ether and maleic anhydride, and ortho-cresol novolac epoxy resins.

The crosslinking agent is not limited, and examples of the crosslinking agent include polyethylene glycol diglycidyl ether (PEGDGE), glutaraldehyde, disuccinimidyl suberate, and succinimidyl-4-(p-maleimidophenyl)butyrate.

A method of layering the second catalyst layer on the electrode base material is also not limited, and examples of the method include a method in which a composition containing the enzyme (b) and NAD(P)H and/or NAD(P) (hereinafter, also referred to as enzyme (b)-containing composition) is applied to the electrode base material on which the first catalyst layer is layered, and dried. The method of applying the enzyme (b)-containing composition is not limited, and examples of the method include the above-described method of applying the enzyme (a1)-containing composition.

The enzyme (b)-containing composition is to contain the enzyme (b) and NAD(P)H and/or NAD(P), and may contain a solvent and a buffer component described above. The enzyme (b)-containing composition may contain the above-described immobilizing agent such as a polymer or a crosslinking agent, and the enzyme (b) may be more strongly immobilized to the electrode base material by the immobilizing agent.

### <First Catalyst Layer>

The enzyme (a1) is to be capable of catalyzing an oxidation-reduction reaction of a redox couple of NAD(P)H and NAD(P) and capable of direct electron exchange, between the enzyme (a1) and the electrode base material, accompanying the oxidation-reduction reaction, and preferably has one flavin mononucleotide (hereinafter, also referred to as FMN) and at least one Fe—S cluster.

The FMN and the Fe—S cluster can transfer electrons, and one of preferred embodiments of the present disclosure is a form in which the enzyme (a1) has the FMN and the Fe—S cluster as cofactors.

The number of Fe—S clusters in the enzyme (a1) is not limited, and is preferably 1 to 10 per molecule. The number per molecule is more preferably 2 to 8, still more preferably 3 to 5.

In the present description, the number of Fe—S clusters per enzyme molecule can be measured with an atomic absorption apparatus utilizing an acetylene combustion method.

The Fe—S cluster includes an iron atom and a sulfur atom, and a cysteine residue in polypeptide is mainly coordinated to the iron atom of the Fe—S cluster.

Specific examples of the Fe—S cluster include a [2Fe-2S] cluster, a [3Fe-4S] cluster, and a [4Fe-4S] cluster.

In a case where the enzyme (a1) contains at least one Fe—S cluster, the type of the cluster is not limited, and the at least one Fe—S cluster preferably includes a [2Fe-2S] cluster and/or a [4Fe-4S] cluster. A form is more preferable in which the at least one Fe—S cluster includes a [2Fe-2S] cluster, a form is still more preferable in which the at least one Fe—S cluster includes a [2Fe-2S] cluster and a [4Fe-4S] cluster, a form is particularly preferable in which the at least one Fe—S cluster includes one [2Fe-2S] cluster and one [4Fe-4S] cluster.

In the present description, the number of Fe—S clusters and the number of FMNs described below in the enzyme each mean the number per enzyme molecule.

In a case where the enzyme (a1) has FMN and an Fe—S cluster, electrons are preferably exchanged from NAD(P)H to the electrode base material via FMN and at least one Fe—S cluster in the enzyme (a1).

The distance between the FMN and the Fe—S cluster is not limited, and the distance between the FMN and at least one Fe—S cluster is preferably 2 nm or less.

The distance between the FMN and the Fe—S cluster is the distance between the nearest neighbor atoms in both the FMN and the Fe—S cluster.

In the present description, the distance between atoms in an enzyme can be calculated on the basis of three-dimensional structure information.

The three-dimensional structure analysis of an enzyme can be performed by cryo-electron microscopy. Information on the three-dimensional structure of an enzyme may be acquired from a database such as PDB.

In a case where the enzyme (a1) has two or more Fe—S clusters, the FMN and the nearest Fe—S cluster preferably have a distance of 2 nm or less. A form is more preferable in which the FMN and an Fe—S cluster being a [2Fe-2S] cluster have a distance of 2 nm or less.

A form is still more preferable in which the FMN and each of two Fe—S clusters have a distance of 2 nm or less.

The FMN and an Fe—S cluster more preferably have a distance of 1.5 nm or less.

In an aspect, the distance is still more preferably 1 nm or less.

In a case where the enzyme (a1) has two or more Fe—S clusters, at least two Fe—S clusters preferably have a distance of 3 nm or less from each other. The distance is more preferably 2.5 nm or less.

In the enzyme (a1), at least one Fe—S cluster is preferably present at a distance of 2 nm or less from the surface of the enzyme (a1). Thus, the redox center in the enzyme and the electrode have a shorter distance to improve the interfacial electron transfer rate between the enzyme and the electrode.

In the present description, the distance to an Fe—S cluster or the like from the surface of the enzyme means the distance between the α carbon of an amino acid residue present on the surface of the enzyme and the atom of the Fe—S cluster or the like.

In the present description, the amino acid residue present on the enzyme surface means an amino acid residue, based on the PDBePISA (http://www.ebi.ac.uk/msd-srv/prot_int/) program, capable of accessing the solvent in the enzyme (a1).

In a case where the enzyme (a1) has two or more Fe—S clusters, at least two Fe—S clusters are preferably present at a distance of 2 nm or less from the surface of the enzyme (a1).

In one of preferred embodiments of the present disclosure, all of the Fe—S clusters in the enzyme (a1) are present at a distance of 2 nm or less from the surface of the enzyme (a1).

One of preferred embodiments of the present disclosure is a form in which the enzyme (a1) has one flavin mononucleotide and one or more Fe—S clusters, the flavin mononucleotide and at least one Fe—S cluster have a distance of 2 nm or less, and at least one Fe—S cluster is present at a distance of 2 nm or less from the surface of the enzyme (a1).

In one of preferred embodiments of the present disclosure, the enzyme (a1) has an Fe—S cluster being a [2Fe-2S] cluster at a distance of 2 nm or less from the surface of the enzyme (a1).

The Fe—S cluster having a distance of 2 nm or less from the FMN and the Fe—S cluster present at a distance of 2 nm or less from the enzyme surface may be the same or different, and are preferably the same.

The enzyme (a1) preferably has a β subunit having one flavin mononucleotide and one or more Fe—S clusters.

The enzyme (a1) preferably has two Fe—S clusters in the β subunit. A form is more preferable in which the enzyme (a1) has an Fe—S cluster being a [2Fe-2S] cluster and an Fe—S cluster being a [4Fe-4S] cluster in the β subunit.

The enzyme (a1) preferably contains a β subunit of a formate dehydrogenase derived from a methanol-assimilating bacterium.

The methanol-assimilating bacterium is not limited, and is preferably a bacterium belonging to the genus Methylorubrum, more preferably Methylorubrum extorquens, still more preferably Methylorubrum extorquens AM1.

SEQ ID NO: 1 shows the amino acid sequence of the α subunit of a formate dehydrogenase derived from Methylorubrum extorquens AM1.

SEQ ID NO: 2 shows the amino acid sequence of the β subunit of the formate dehydrogenase derived from Methylorubrum extorquens AM1.

The enzyme (a1) may further have an α subunit having at least one Fe—S cluster.

The type of the Fe—S cluster in the α subunit is not limited, and the Fe—S cluster is preferably a [4Fe-4S] cluster and/or a [2Fe-2S] cluster.

The number of Fe—S clusters in the α subunit is not limited, and is preferably 1 to 5, more preferably 1 to 4, particularly preferably 2 to 4, and most preferably 4.

One of preferred embodiments of the present disclosure is a form in which the a subunit has three Fe—S clusters being [4Fe-4S] clusters and one Fe—S cluster being a [2Fe-2S] cluster.

The α subunit preferably further has tungsten pterin, which is a pterin complex containing tungsten as a central metal.

Tungsten pterin can play a role as an active center that catalyzes an oxidation-reduction reaction using, for example, formic acid, carbon dioxide, or the like as a substrate.

In a case where the enzyme (a1) has tungsten pterin, electrons obtained from a substrate such as formic acid can be transferred to the electrode base material, and electrons transferred from the electrode base material can be transferred to a substrate such as carbon dioxide.

In a case where the enzyme (a1) has a β subunit and an α subunit, the positional relationship between the subunits is not limited, and at least one Fe—S cluster in the β subunit and at least one Fe—S cluster in the α subunit preferably have a distance of 2 nm or less. The distance is more preferably 1 nm or less.

The enzyme (a1) may contain an α subunit of a formate dehydrogenase derived from a methanol-assimilating bacterium, or may contain no α subunit of such a formate dehydrogenase.

The enzyme (a1) preferably has an average molecular weight of 50000 to 500000. Thus, the electron transfer rate between the enzyme (a1) and the electrode base material can be further improved.

In a case where the enzyme (a1) contains only the β subunit, the average molecular weight is more preferably 50000 to 100000, still more preferably 60000 to 80000.

In a case where the enzyme (a1) contains the α subunit, the average molecular weight is more preferably 150000 to 300000, still more preferably 160000 to 200000.

The average molecular weight of the enzyme can be measured by gel filtration chromatography.

The enzyme (a1) preferably has an amino acid sequence having 70% or more sequence identity to the amino acid sequence set forth in SEQ ID NO: 2.

The sequence identity is more preferably 80% or more, still more preferably 90% or more.

The sequence identity of the amino acid sequence can be calculated by the BLAST-P program provided by NCBI.

The surface charge of the enzyme (a1) is not limited, and a portion located at a distance of 2 nm or less from the active center is preferably positively or negatively charged. In this case, for example, the surface of the electrode base material is modified with a surface-modifying group having a charge opposite to the surface charge of the enzyme (a1), and thus the enzyme (a1) can be immobilized so that the active center of the enzyme (a1) is aligned on the electrode base material. Thus, the electron transfer rate between the enzyme (a1) and the electrode base material can be further enhanced.

A form is more preferable in which in the surface of the enzyme (a1), the portion located at a distance of 2 nm or less from the active center has a negative surface charge. A form is particularly preferable in which a portion located at a distance of 2 nm or less from an Fe—S cluster contained in the enzyme (a1) has a negative surface charge.

The enzyme (a1) may be any enzyme as long as it catalyzes an oxidation-reduction reaction of a redox couple of NAD(P)H and NAD(P), and is preferably an enzyme having catalytic activity in an oxidation reaction of NAD(P)H.

The enzyme activity in an oxidation reaction of NAD(P)H is not limited, and is preferably 0.1 units or more per milligram of the enzyme.

The enzyme activity is more preferably 0.5 units or more, still more preferably 1.0 units or more, particularly preferably 1.5 units or more.

The enzyme activity can be measured with a solution enzyme activity evaluation method using NADH as a substrate.

The amount of the enzyme (a1) included in the first catalyst layer is not limited, and is preferably 0.05 pmol/cm² to 1000 pmol/cm² with respect to the surface area of the electrode base material. The amount is more preferably 0.5 pmol/cm² to 100 pmol/cm², still more preferably 5 pmol/cm² to 10 pmol/cm².

The origin and the like of the enzyme (a1) are not limited as long as the enzyme (a1) has the above-described catalytic function. For example, the origin may be a biologically derived molecule extracted from a plant, an animal, a microorganism, or the like, or may be a genetically engineered or chemically synthesized product. The enzyme (a1) may be a naturally derived product prepared with a technique of isolating and purifying an appropriate protein from any organism such as a naturally occurring bacterium, yeast, animal, or plant, or may be a product produced as a recombinant with a genetic engineering technique, or a chemically synthesized product.

An organism, such as a microorganism, an organelle, or a cell, including the enzyme (a1) itself may be used in the first catalyst layer. Alternatively, a crude product from such an organism may be used in the first catalyst layer.

In the case of producing the enzyme (a1) as a recombinant with a genetic engineering technique, a method can be used that is commonly used in the art.

Examples of the method include a method in which an appropriate probe DNA is synthesized based on the amino acid sequence of the formate dehydrogenase derived from Methylorubrum extorquens AM1 (the amino acid sequences set forth in SEQ ID NOs: 1 and 2) and the probe DNA is used to select a formate dehydrogenase gene from a library of chromosomal DNAs or cDNAs, or a method in which an appropriate primer DNA is prepared based on the amino acid sequence, a DNA containing a target gene fragment is amplified by an appropriate polymerase chain reaction (PCR method) such as a 5′RACE method or a 3′RACE method, and the obtained DNA fragments are linked to obtain the entire length of the target gene.

The gene encoding the enzyme (a1) may be linked to or inserted into various vectors, or may be incorporated into a chromosome or a genome. In the case of using a vector, cloning into the vector can be performed using a commercially available kit such as TA Cloning Kit (Invitrogen) or In-Fusion HD Cloning Kit (Clontech Laboratories, Inc.); a commercially available plasmid vector DNA such as pUC119 (Takara Bio Inc.), pUC18 (Takara Bio Inc.), pBR322 (Takara Bio Inc.), pBluescript SK+(Stratagene), pYES2/CT (Invitrogen), or pET21a (+); or a commercially available bacteriophage vector DNA such as λEMBL3 (Stratagene). A homologous recombination vector can also be used such as pK18mobsacB (Schaefer et a1., Gene, vol. 45, p. 69-73 (1994)) or pCM1682 (H. Iguchi et a1., Environ. Microbiol. Rep. 10 (2018) 634-643).

The recombinant DNA thus obtained can be used to transform a host organism, for example, Methylorubrum extorquens or Escherichia coli such as E. coli DE3, E. coli JM109 (Takara Bio Inc.), or E. coli DH5α (Takara Bio Inc.).

The transformant obtained as described above is preferably cultured under conditions that allow expression of the introduced gene, and from the culture of the transformant, the enzyme (a1) is preferably isolated and purified.

The method of isolating and purifying the enzyme (a1) is not limited, and an ordinary method of isolating and purifying a protein can be used.

Examples of the isolation and purification method include known isolation and purification techniques such as ammonium sulfate precipitation, dialysis, SDS-PAGE electrophoresis, gel filtration, and various types of chromatography such as hydrophobicity chromatography, anion chromatography, cation chromatography, and affinity chromatography, and these techniques can be used singly or in appropriate combination. In particular in the case of using affinity chromatography, the enzyme (a1) is preferably expressed as a fusion protein with a tag peptide such as a histidine tag (His-Tag) to utilize affinity for such a tag peptide.

In the case of producing the enzyme (a1) as a recombinant, for example, a mutation may be introduced into the amino acid sequence, set forth in SEQ ID NO: 1, of the formate dehydrogenase derived from Methylorubrum extorquens AM1.

In introduction of a mutation, a person skilled in the art can predict, for example, a mutation that alters the surface charge while maintaining the enzyme activity, on the basis of the information on the three-dimensional structure of the formate dehydrogenase, and the like, and properties of amino acids.

### <Second Catalyst Layer>

The NAD(P)H or NAD(P) dependent oxidoreductase (b) (hereinafter, also referred to as enzyme (b)) included in the second catalyst layer may be any enzyme as long as it catalyzes an oxidation-reduction reaction of a substrate using NAD(P)H or NAD(P) as an electron acceptor.

In a case where the electrode of the present disclosure is an anode, the enzyme (b) is to be an oxidase (dehydrogenase) of the substrate, and in a case where the electrode of the present disclosure is a cathode, the enzyme (b) is to be a reductase of the substrate.

The substrate is not limited, and examples of the substrate include alcohols, saccharides, fats, polyamino acids such as peptides and proteins, and organic acids. These substrates can be used singly or in combination of two or more kinds thereof.

The alcohols are not limited, and examples thereof include monohydric alcohols such as methanol, ethanol, n-propyl alcohol, iso-propyl alcohol, n-butyl alcohol, sec-butyl alcohol, and tert-butyl alcohol, and polyhydric alcohols such as ethylene glycol, diethylene glycol, and glycerol (glycerin).

The saccharides are not limited, and examples thereof include glucose, glucose-1, D-glucose, L-glucose, glucose-6-phosphate, lactate, lactate-6-phosphate, D-lactate, L-lactate, fructose, galactose-1, galactose, aldose, sorbose, and mannose.

The organic acids are not limited, and examples thereof include intermediate products of sugar metabolism, such as glucose-6-phosphate, fructose-6-phosphate, fructose-1,6-bisphosphate, 1,3-bisphosphoglyceric acid, 3-phosphoglyceric acid, 2-phosphoglyceric acid, phosphoenolpyruvic acid, pyruvic acid, acetyl-CoA, citric acid, cis-aconitic acid, isocitric acid, oxalosuccinic acid, 2-oxoglutaric acid, succinyl-CoA, succinic acid, fumaric acid, L-malic acid, and oxaloacetic acid.

The fats are not limited as long as they are an ester of a fatty acid and glycerin, and the fatty acid that constitutes the fat is also not limited and may be a saturated fatty acid, a monounsaturated fatty acid, or a polyunsaturated fatty acid.

The substrate is more preferably an alcohol or a saccharide, still more preferably glycerol.

The enzyme (b) is preferably an oxidase.

Examples of the enzyme (b) that is more preferable include glycerol dehydrogenase, glucose dehydrogenase, a series of enzymes of an electron transport system, ATP synthase, and enzymes involved in sugar metabolism (such as hexokinase, glucose phosphate isomerase, phosphofructokinase, fructose diphosphate aldolase, triose phosphate isomerase, glyceraldehyde phosphate dehydrogenase, phosphoglycerate mutase, phosphopyruvate hydratase, pyruvate kinase, L-lactate dehydrogenase, D-lactate dehydrogenase, pyruvate dehydrogenase, citrate synthase, aconitase, isocitrate dehydrogenase, 2-oxoglutarate dehydrogenase, succinyl-CoA synthetase, succinate dehydrogenase, fumarase, and malonate dehydrogenase). These enzymes can be used singly or in combination of two or more kinds thereof.

Among them, glycerol dehydrogenase and glucose dehydrogenase are preferable.

In an aspect, the second catalyst layer may include a plurality of the enzymes (b), and the substrate can be decomposed stepwise by the plurality of the enzymes (b).

The origin and the like of the enzyme (b) are not limited as long as the enzyme (b) has the above-described catalytic function. The enzyme (b) may be a naturally derived product purified with a technique of isolating and purifying an appropriate protein from a naturally occurring organism, or may be a product produced as a recombinant with a genetic engineering technique, or a chemically synthesized product. Alternatively, a commercially available product may be used.

Examples of the method of producing the enzyme (b) as a recombinant with a genetic engineering technique include a method similar to the method of producing the enzyme (a1).

An organism, such as a microorganism, an organelle, or a cell, including the enzyme (b) itself may be used in the second catalyst layer. Alternatively, a crude product from such an organism may be used in the second catalyst layer.

The amount of the enzyme (b) included in the second catalyst layer is not limited, and is preferably 0.01 mol % to 10000 mol % with respect to 100 mol % of the enzyme (a1). The amount is more preferably 0.1 mol % to 1000 mol %, still more preferably 1 mol % to 100 mol %.

The amount of NAD(P)H or NAD(P) included in the second catalyst layer is not limited, and the total amount of NAD(P)H and NAD(P) is preferably 0.5 mol % to 50000 mol % with respect to 100 mol % of the enzyme (a1). The amount is more preferably 5 mol % to 5000 mol %, still more preferably 50 mol % to 500 mol %.

### <Electrode Base Material>

The electrode base material included in the enzyme electrode of the present disclosure is a conductive base material that can be connected to an external circuit and can transmit electrons. The material, shape, and the like of the electrode base material are not limited as long as the electrode base material has the above properties.

The material of the electrode base material may be any conductive material, and examples of the material include carbon materials such as carbon cloth, carbon paper, graphite, glassy carbon, activated carbon, carbon black, and carbon nanotubes, metals such as gold, platinum, copper, palladium, titanium, aluminum, silver, and nickel and alloys thereof, and conductive oxides such as SnO₂, In₂O₃, WO₃, and TiO₂.

The electrode base material may include a single layer of one of these materials or may include a laminated structure of two or more layers.

The carbon materials are preferably activated carbon, carbon black, and carbon nanotubes.

Using a carbon material as the conductive material can further improve the conductivity with the electrode.

The carbon material is preferably in the form of particles.

One of preferred embodiments of the present disclosure is a form in which the enzyme electrode of the present disclosure includes at least one kind of carbon particles selected from the group consisting of activated carbon, carbon black, and carbon nanotubes.

The metals are preferably gold, platinum, copper, palladium, titanium, and the like.

One of preferred embodiments of the present disclosure is a form in which the enzyme electrode of the present disclosure includes at least one selected from the group consisting of gold, platinum, copper, palladium, and titanium.

In the case of using, for example, two or more of the above-described conductive materials in the electrode base material, a binder such as a polymer may be used.

The polymer is not limited, and a polymer containing a fluorine atom, such as polyvinylidene fluoride (PVDF) or polyvinyl fluoride (PVF), may be used. Alternatively, a copolymer thereof or a copolymer of such a monomer with ethylene, styrene, or the like may be used. Examples of the polymer further include polystyrene, polyethylene, polypropylene, hydrophilic polymers such as polyacrylic acid, polylysine, and carboxymethylcellulose, and conductive polymers such as polyaniline, polypyrrole, and polyaniline sulfonic acid as a derivative thereof.

Among them, conductive polymers are preferable, and polyaniline and polypyrrole are more preferable.

One of preferred embodiments of the present disclosure is a form in which the enzyme electrode of the present disclosure includes at least one selected from the group consisting of polyaniline and polypyrrole.

The enzyme electrode of the present disclosure preferably includes carbon particles, a metal, or a conductive polymer as a conductive electrode material.

One of preferred embodiments of the present disclosure is a form in which the enzyme electrode of the present disclosure includes at least one selected from the group consisting of carbon particles, metals, and conductive polymers.

The electrode base material may have a flat base material surface or may have irregularities and pores on the base material surface, and preferably has pores. Thus, the first catalyst layer and the second catalyst layer are more sufficiently fixed to the electrode base material.

The size of each pore is not limited, and is preferably 1 nm to 100 nm. The size is more preferably 2 nm to 10 nm.

In the electrode base material, the base material surface may be covered or may be not covered with a surface-modifying group, and is preferably covered.

The surface-modifying group is preferably capable of interaction with the enzyme (a1) by an intermolecular force, a hydrogen bond, a Coulomb force, or the like, or capable of covalent bond with the enzyme (a1).

The surface-modifying group is more preferably capable of interaction with the enzyme (a1) by a Coulomb force, still more preferably has a charge opposite to the surface charge of the enzyme (a1).

In a case where the enzyme (a1) has a positive surface charge, the surface-modifying group is preferably anionic. In a case where the enzyme (a1) has a negative surface charge, the surface-modifying group is preferably cationic.

The surface-modifying group particularly preferably has a charge opposite to the surface charge of a portion, on the surface of the enzyme (a1), located at a distance of 2 nm or less from an Fe—S cluster contained in the enzyme (a1).

The surface-modifying group is not limited, and examples of the surface-modifying group include functional groups and reactive groups such as an amine group, a sulfonic acid group, a sulfuric acid group, a phosphoric acid group, a sulfhydryl group, a carboxyl group, groups of salts thereof, a thiol group, a hydroxyl group, an azide group, an azo group, a nitro group, a nitrile group, a cyano group, an allenic group, an isonitrile group, a urea group, an aldehyde group, a ketone group, an NHS ester, an imide ester, maleimide, pyridyldithiol, allyl azide, haloacetate, isocyanate, carbodiimide, allyl azide, diazirine, a hydrazide, psoralen, iodo, pyridine disulfide, and vinyl sulfone.

The surface-modifying group preferably has an anionic group or a cationic group.

The anionic group is a group having an anion or a group capable of releasing a hydrogen ion to form an anion, and examples thereof include a carboxyl group, a sulfonic acid group, a sulfuric acid group, a phosphoric acid group, and groups of salts thereof.

The cationic group is a group having a cation or a group capable of accepting a hydrogen ion to form a cation, and examples thereof include primary to tertiary amino groups, neutralized products of primary to tertiary amino groups with an acid such as hydrochloric acid or acetic acid, and amine groups such as quaternary ammonium bases.

A surface-modifying group-containing compound to be used for modifying the surface of the electrode base material is preferably a compound having an anionic group or a cationic group and having a structure capable of interaction with the surface of the electrode base material.

In a case where the surface of the electrode base material is a carbon material, the surface-modifying group-containing compound preferably has a structure capable of π-π interaction with the carbon material. Examples of the structure capable of π-π interaction include structures derived from an aromatic compound or a heterocyclic compound. The aromatic compound may have a hetero atom.

The aromatic compound is more preferably a polycyclic aromatic compound.

The surface-modifying group-containing compound is preferably an aromatic compound having an anionic group or a cationic group, or a heterocyclic compound having an anionic group or a cationic group.

Examples of the polycyclic aromatic compound include pyrene, coronene, chrysene, naphthacene, pentacene, picene, perylene, anthracene, phenanthrene, fluorene, naphthalene, fluoranthene, acenaphthene, acenaphthylene, triphenylene, and derivatives thereof.

Examples of the aromatic compound or heterocyclic compound having a hetero atom include terthiophene, tetraphenylbenzidine, tetraphenylnaphthacene, benzothiophene, thiophene, pyrrole, carbazole, phenanthroline, phenylpyridine, quinoline, triphenylamine, diphenylamine, oxazole, oxadiazole, quinacridone, flucrenone, phthalocyanine, spiropyran, viologen, spiroperimidine, benzoic acid, benzophenone, phenylamine, diphenyl ether, diphenyl sulfide, diphenyl sulfone, bisphenol, anthraquinone, phosphonium compounds, fluorecene, rhodamine, coumarin, cyanine, and derivatives thereof.

The surface-modifying group-containing compound is preferably a pyrene derivative. The pyrene derivative preferably has, for example, a functional group or a reactive group described above, and more preferably has an anionic group or a cationic group.

Specific examples of the pyrene derivative include 1-pyrenemethylamine, 1-aminopyrene, dimethyl-pyrene-1-yl-methylamine, and diethyl-pyrene-1-yl-amine.

The functional group or the reactive group and pyrene may be separated from each other by a spacer that is an alkyl group, polyethylene glycol, or the like. These modifications such as the functional group, the reactive group, and the spacer may be bonded to carbon at any position in pyrene.

The amount of the surface-modifying group-containing compound used is not limited, and is preferably 0.05 pmol/cm² to 1000 pmol/cm² with respect to the surface area of the electrode base material. The amount is more preferably 0.5 pmol/cm² to 100 pmol/cm², still more preferably 5 pmol/cm² to 10 pmol/cm².

Hereinafter, an embodiment of the enzyme electrode of the present disclosure will be described with reference to the drawings.

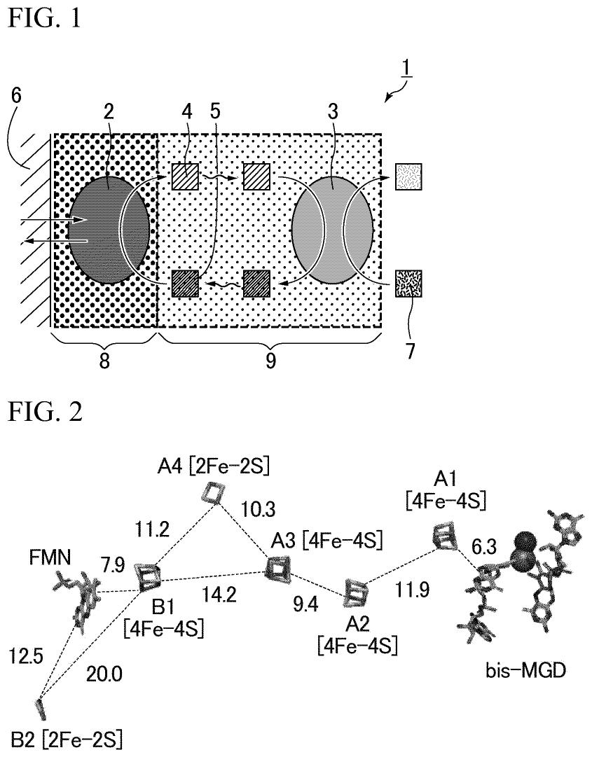

FIG. 1 is a schematic diagram of an embodiment of the enzyme electrode of the present disclosure. An enzyme electrode 1 includes an electrode base material 6, a first catalyst layer 8, and a second catalyst layer 9, the first catalyst layer 8 includes an enzyme (a1) 2 capable of catalyzing an oxidation-reduction reaction of a redox couple of NAD(P)H 5 and NAD(P) 4 and capable of direct electron exchange, between the enzyme (a1) 2 and the electrode base material 6, accompanying the oxidation-reduction reaction, and the second catalyst layer 9 includes an enzyme (b) 3 and NAD(P)H 5 and/or NAD(P) 4.

In a case where the enzyme electrode 1 of the present disclosure is an anode, NAD(P) 4 receives electrons generated by oxidation of a substrate 7 catalyzed by the enzyme (b) 3 and becomes NAD(P)H 5, the NAD(P)H 5 is oxidized by the enzyme (a1) 2 to generate electrons, and the electrons can be directly transferred to the electrode base material 6 by the enzyme (a1) 2.

[Oxidoreductase (a2)]

The present disclosure is also an enzyme capable of catalyzing an oxidation-reduction reaction of a redox couple of NAD(P)H and NAD(P) (hereinafter, also referred to as enzyme (a2)), the enzyme includes one flavin mononucleotide and two Fe—S clusters, the one flavin mononucleotide and at least one of the two Fe—S clusters have a distance of 2 nm or less, and at least one of the two Fe—S clusters is present at a distance of 2 nm or less from a surface of the enzyme.

The enzyme (a2) is not limited as long as it has the above configuration, and is preferably capable of direct electron exchange, between the enzyme (a2) and an electrode base material, accompanying the oxidation-reduction reaction when used in an electrode.

In the enzyme (a2), the types of the two Fe-s clusters are not limited, and the two Fe—S clusters preferably include a [2Fe-2S] cluster and/or a [4Fe-4S] cluster. A form is more preferable in which the two Fe—S clusters include a [2Fe-2S] cluster, a form is still more preferable in which the two Fe—S clusters include one [2Fe-2S] cluster and one [4Fe-4S] cluster.

In the enzyme (a2), the FMN and the nearest Fe—S cluster preferably have a distance of 2 nm or less. A form is more preferable in which the FMIN and an Fe—S cluster being a [2Fe-2S] cluster have a distance of 2 nm or less.

A form is still more preferable in which the FMN and each of the two Fe—S clusters have a distance of 2 nm or less.

The average molecular weight of the enzyme (a2) is not limited, and is preferably 50000 to 100000. Thus, when the enzyme (a2) is used in an electrode, the electron transfer rate between the enzyme (a2) and the electrode base material is further improved. The average molecular weight of the enzyme (a2) is more preferably 60000 to 80000.

The enzyme (a2) preferably has 70% or more sequence identity to the amino acid sequence set forth in SEQ ID NO: 2.

The sequence identity is more preferably 80% or more, still more preferably 90% or more.

The sequence identity of the amino acid sequence can be calculated by the BLAST-P program provided by NCBI.

The surface charge of the enzyme (a2) is not limited, and a portion located at a distance of 2 nm or less from the active center is preferably positively or negatively charged. In this case, in the case of using the enzyme (a2) in an electrode, for example, the surface of the electrode base material is modified with a surface-modifying group having a charge opposite to the surface charge of the enzyme (a2), and thus the enzyme (a2) can be immobilized so that the active center of the enzyme (a2) is aligned on the electrode base material. Thus, the electron transfer rate between the enzyme (a2) and the electrode base material can be further enhanced.

A form is more preferable in which in the surface of the enzyme (a2), the portion located at a distance of 2 nm or less from the active center has a negative surface charge. A form is particularly preferable in which a portion located at a distance of 2 nm or less from an Fe—S cluster contained in the enzyme (a2) has a negative surface charge.

The origin and the like of the enzyme (a2) are not limited as long as the enzyme (a2) has the above-described configuration. The enzyme (a2) may be a naturally derived product purified with a technique of isolating and purifying an appropriate protein from a naturally occurring organism, or may be a product produced as a recombinant with a genetic engineering technique, or a chemically synthesized product.

In the case of producing the enzyme (a2) as a recombinant with a genetic engineering technique, the enzyme (a2) can be produced, for example, using a method similar to the above-described method of producing the enzyme (a1) on the basis of the amino acid sequence of the β subunit of the formate dehydrogenase derived from Methylorubrum extorquens AM1 (the amino acid sequence set forth in SEQ ID NO: 2).

In the case of using the enzyme (a2) in an electrode or the like, an organism, such as a microorganism, an organelle, or a cell, including the enzyme (a2) itself may be used. Alternatively, a crude product from such an organism may be used.

In the case of producing the enzyme (a2) as a recombinant, as described for the enzyme (a1), for example, a mutation may be introduced into the amino acid sequence, set forth in SEQ ID NO: 2, of the β subunit of the formate dehydrogenase derived from Methylorubrum extorquens AM1.

The enzyme (a2) can be suitably used as an electrode of a fuel cell or the like.

A form in which the enzyme (a1) in the enzyme electrode of the present disclosure is the enzyme (a2) is also one of preferred embodiments of the present disclosure.

### [Biosensor]

The present disclosure is also a biosensor including the enzyme electrode of the present disclosure.

The biosensor of the present disclosure preferably includes the enzyme electrode of the present disclosure as a working electrode, and includes a counter electrode of the enzyme electrode.

In the biosensor, a measurement sample is brought into contact with the biosensor to cause an oxidation-reduction reaction between the enzyme (b) included in the enzyme electrode of the present disclosure and the substance to be measured, and a current generated by the oxidation-reduction reaction is detected to perform measurement. Such a response current value can be used to determine the presence or absence, or the concentration of a substrate in the sample.

Examples of the measurement method using the biosensor of the present disclosure include commonly used methods such as chronoamperometry in which an oxidation current or a reduction current is measured, coulometry, and cyclic voltammetry.

### [Bioreactor]

The present disclosure is also a bioreactor including the enzyme electrode of the present disclosure.

The bioreactor of the present disclosure is not limited as long as the enzyme electrode of the present disclosure acts as a reaction site with a reactant, and in an aspect, the enzyme electrode of the present disclosure is preferably installed in a column reactor.

In the above aspect, when a solution containing the reactant flows in the column reactor and thus becomes in contact with the enzyme electrode, the enzyme (b) included in the enzyme electrode of the present disclosure causes an enzyme reaction, and thus a product is obtained from the reactant.

The reactant to be applied to the bioreactor is not limited as long as it is a substrate to be oxidized or reduced by the enzyme (b). Specific examples of the reactant include the above-described substrates.

### [Biofuel Cell]

The present disclosure is also a biofuel cell including the enzyme electrode of the present disclosure.

The enzyme electrode of the present disclosure in the biofuel cell may be an anode or a cathode, and is preferably an anode.

The biofuel cell of the present disclosure includes the enzyme electrode of the present disclosure. The biofuel cell is not limited as long as it includes an anode and a cathode that are connected by an external circuit, and preferably includes a diaphragm that separates the anode and the cathode.

In an aspect, the biofuel cell of the present disclosure preferably includes an anode including the enzyme electrode of the present disclosure, a cathode, and a diaphragm that separates the anode and the cathode.

Examples of a catalyst usable as the cathode in the biofuel cell according to an embodiment of the present disclosure include enzyme catalysts such as multi-copper enzymes such as pyruvate oxidase, ascorbate oxidase, and laccase and metal catalysts such as platinum. In a case where the enzyme catalytic mechanism is used for a reaction on the cathode side, the biofuel cell may be formed so that the enzyme is preferably immobilized to the electrode base material, or preferably not immobilized, and supplied as an enzyme solution onto an appropriate electrode base material. At this time, as the electrode base material, the electrode base material described for the enzyme electrode of the present disclosure can be used in the same manner.

The material, the shape, and the like of the diaphragm are not limited as long as the diaphragm has ion conductivity so as to transmit protons and the like, and has a property so as not to transmit constituent components on the negative electrode side and constituent components on the positive electrode side other than ions such as protons. For example, a cellulose membrane or the like can be used, or a solid electrolyte membrane can be used. Examples of the solid electrolyte membrane include, but are not limited to, solid membranes having an ion exchange function, such as organic polymers having a strong acid group such as a sulfo group, a phosphate group, a phosphone group, or a phosphine group, a weak acid group such as a carboxy group, and a polar group. Specifically, a cellulose membrane or a perfluorocarbon sulfonic acid (PFS)-based resin membrane can be used, and examples of the PFS-based resin membrane include Nafion (registered trademark), which is a copolymer of tetrafluoroethylene and perfluoro[2-(fluorosulfonylethoxy)propylvinyl ether.

### EXAMPLES

Hereinafter, examples will be shown for more specifically describing the present disclosure. Note that the present disclosure is not limited only to these Examples.

### Preparation Example 1: Enzyme (a1) (rFoDH1)

A recombinant of the formate dehydrogenase derived from Methylorubrum extorquens AM1 (hereinafter, also referred to as rFoDH1) was prepared as follows.

A formate dehydrogenase gene-disrupted strain of Methylorubrum extorquens AM1 was prepared. A formate dehydrogenase gene, a methanol dehydrogenase subunit 1 precursor gene as a promoter, and a His-tag sequence for enzyme purification were inserted into a plasmid for genomic DNA transformation (pCM1682), and the obtained plasmid and the disrupted strain were mixed and electroporated to perform transformation. The transformed strain was cultured and then subjected to cell disruption to obtain a suspension, the suspension was centrifuged, and the obtained supernatant was subjected to a Ni-NTA Agarose (manufactured by QIAGEN) column to purify the enzyme, and thus a recombinant (rFoDH1) was obtained.

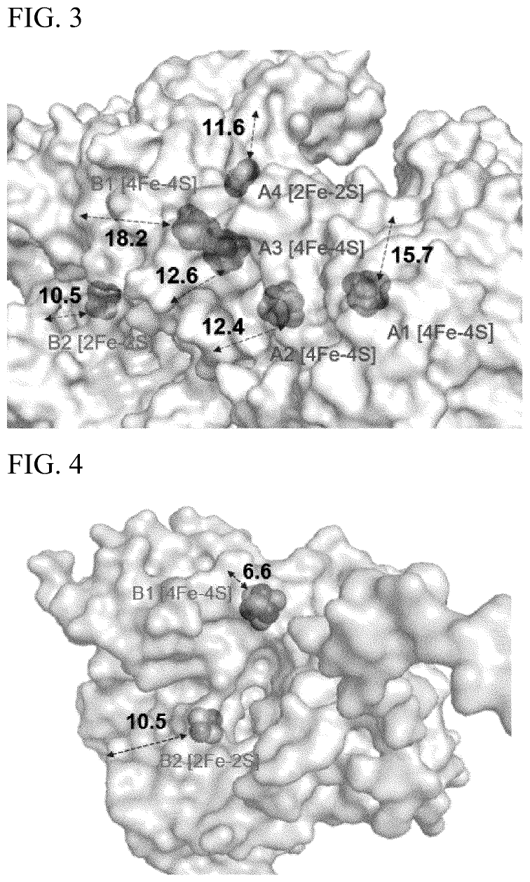

FIG. 2 shows the distances between cofactors of the rFoDH1 on the basis of the three-dimensional structure analysis of the rFoDH1, and FIG. 3 shows the distances from each cofactor to the protein surface.

In FIGS. 2 and 3, for the α subunit of the rFoDH1, the Fe—S clusters being [4Fe-4S] clusters were denoted by A1, A2, and A3, and the Fe—S cluster being a [2Fe-2S] cluster was denoted by A4, and in FIGS. 2 to 4, for the β subunit of the rFoDH1 or for FoDH1β, the Fe—S cluster being a [4Fe-4S] cluster was denoted by B1, and the Fe—S cluster being a [2Fe-2S] cluster was denoted by B2.

The unit of each numerical value in FIGS. 2 to 4 is angstrom.

### Preparation Example 2: Enzyme (a2) (rFoDH1β (Me))

A β subunit of the formate dehydrogenase derived from Methylorubrum extorquens AM1 (hereinafter, also referred to as FoDH1β) was prepared as follows.

A formate dehydrogenase gene-disrupted strain of Methylorubrum extorquens AM1 was prepared. A formate dehydrogenase p subunit gene, a methanol dehydrogenase subunit 1 precursor gene, and a His-tag sequence were inserted into a plasmid for genomic DNA transformation (pCM1682), and the obtained plasmid and the disrupted strain were mixed and electroporated to perform transformation. The transformed strain was cultured and then subjected to enzyme purification in the same manner as in Preparation Example 1, and thus a recombinant (rFoDH1β (Me)) was obtained.

### Preparation Example 3: Enzyme (a2) (rFoDH1β (Ec))

A β subunit of the formate dehydrogenase derived from Methylorubrum extorquens AM1 (hereinafter, also referred to as rFoDH1β (Ec)) was prepared by heterologous expression by E. coli as follows.

A CAT was added to the 5′ end of the gene encoding the FoDH1β, the termination codon at the 3′ end was substituted with CTCGAG, and the resulting product was introduced into a pET21a(+) vector using restriction enzymes NdeI and XhoI to obtain a plasmid pET21a-FoDH1β. The obtained pET21a-FoDH1β was introduced into an E. coli ArcticExpress (DE3) strain for protein expression (Agilent 230192) in accordance with the attached protocol to construct a plasmid-introduced strain.

In pre-culture, the plasmid-introduced strain was inoculated into 5 ml of a TB medium (containing an antibiotic (100 g/ml ampicillin+20 g/ml gentamicin)) containing iron citrate at a final concentration of 0.3 mM, and cultured overnight at 37° C. at 200 rpm. The obtained culture medium was diluted 100 times and cultured in 20 ml of a TB medium (antibiotics containing 0.3 mM iron citrate) in a 50 ml baffle-free flask at 37° C. at 200 rpm for 3.5 hours. Then, the entire flask was put into ice water to cool the culture medium, isopropyl-β-D(−)-thiogalactopyranoside (IPTG) was added so that the final concentration was 0.4 mM, and then the mixture was cultured overnight at 15° C. at 200 rpm.

The obtained culture medium was subjected to cell disruption to obtain a suspension, the suspension was centrifuged to obtain a supernatant, the supernatant was purified by immobilized metal affinity chromatography (TALON (registered trademark) 2 ml Disposable Gravity Column manufactured by Takara Bio Inc.) and subjected to buffer exchange with 100 mM Tris-HCl (pH 8.0) using a centrifugal ultrafiltration filter unit Vivaspin (registered trademark) Turbo15 10,000 MWCO (Sartorius) to obtain rFoDH1β (Ec).

### <Measurement of NADH Oxidation Activity>

The NADH oxidation activity was measured with the following method for the rFoDH1, the rFoDH1β (Me), and the rFoDH1β (Ec) obtained in Preparation Examples 1 and 2.

The measurement was performed using a cuvette having an optical path length of 1 cm in a 100 mM potassium phosphate buffer (pH 7.0) containing 0.2 mM 2,6-dichlorophenolindophenol sodium salt (manufactured by MP Biomedicals, hereinafter also referred to as DCIP) and 1 mM NADH at room temperature (25±2° C.). The change in absorbance at 600 nm accompanying reduction of DCIP was measured, and the enzyme activity was calculated from the molar extinction coefficient at 600 nm (20.6 mM⁻¹ cm⁻¹ at pH 7.0 (J. McD. Armstrong, Biochim. Biophys. Acta 86 (1964) 194-197)). For protein quantification, BCA Protein Assay (manufactured by Thermo Fisher Scientific Inc.) was used.

The results of the measurement of NADH oxidation showed that the rFoDH1, the rFoDH1β (Me), and the rFoDH1β (Ec) had a specific activity of 4.1±0.6 U/mg, 3.0±0.7 U/mg, and 24.7±1.7 U/mg, respectively.

### Preparation Example 4: Enzyme Electrode 1 Including Enzyme (a1) (rFoDH1)

A glassy carbon electrode (3 mmΦ, manufactured by BAS Inc., hereinafter also referred to as GCE) was polished with alumina particles having outer diameters of 1.0 μm and 0.05 μm, and then ultrasonically washed with ultrapure water. Next, 10 mg of multi-walled carbon nanotubes (manufactured by Sigma-Aldrich, outer diameter: 10±1 nm, length: 3 to 6 μm, hereinafter also referred to as MWCNTs) were added to 1-methylpyrrolidone (NMP), then the suspension was sonicated for 2 hours, and 10 μL of the dispersion of the uniformly dispersed MWCNTs was added dropwise to the GCE and dried at 70° C. to obtain a CNT/GCE electrode. The CNT/GCE was immersed in an N,N-dimethylformamide (DMF) solution containing 10 mM 1-pyrenemethylamine hydrochloride (manufactured by Sigma-Aldrich, hereinafter also referred to as PyNH₂) and allowed to stand at room temperature for 1 hour to obtain a PyNH₂/CNT/GCE electrode. The prepared PyNH₂/CNT/GCE was washed with DMF and ultrapure water, and then 15 μL of a 1 mg/mL rFoDH1 solution (dissolved in a 1 M potassium phosphate buffer (pH 8.0)) was added dropwise onto the electrode, the electrode was held under water vapor saturation conditions at 4° C. for 1 hour to immobilize the enzyme, and thus an enzyme electrode 1 was obtained.

### Preparation Example 5: Enzyme Electrode 2 Including Enzyme (a2) (rFoDH1β (Me))

An enzyme electrode 2 was obtained in the same manner as in Preparation Example 3 except that a 1 mg/mL FoDH1B solution (dissolved in a 1 M potassium phosphate buffer (pH 8.0)) was used instead of the rFoDH1 solution.

### Test Example 1: CV Measurement 1

Cyclic voltammetry (CV) measurement was performed using the enzyme electrodes 1 and 2 obtained in Preparation Examples 4 and 5.

The measurement apparatus and the measurement conditions were as follows.
Measurement apparatus: Electrochemical analyzer ALS660E (manufactured by BAS Inc.)
Solution: 1 M potassium phosphate buffer (pH 8.0), 50 mM NADH, 50 mM NAD⁺
Temperature: 25° C.
Atmosphere: Ar
Rotation speed: 100 rpm
Voltage sweep rate: 5 mVs⁻¹

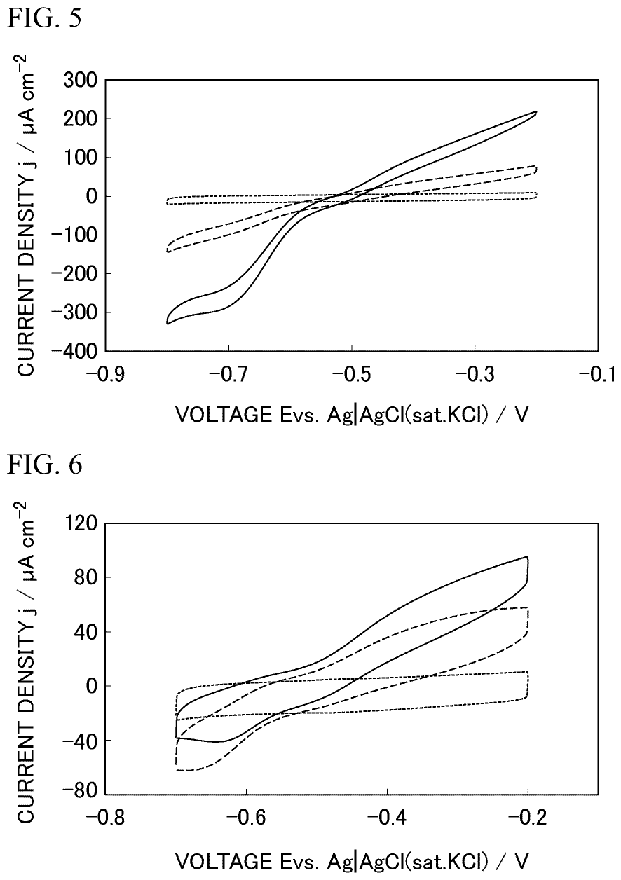

FIG. 5 shows the measurement results. In FIG. 5, the broken line shows the result of the enzyme electrode 1, and the solid line shows the result of the enzyme electrode 2. The dotted line shows the result of measurement of the electrode 2 under a condition without NADH and NAD⁺.

In the enzyme electrodes 1 and 2 in CV measurement 1, an oxidation wave and a reduction wave were observed, and a DET reaction was confirmed. In the enzyme electrode 2 using the FoDH1β, a larger catalytic current density was obtained. This result is considered to be obtained by an increase in the effective amount of the enzyme adsorbed due to the enzyme size.

### Test Example 2: CV Measurement 2

Cyclic voltammetry (CV) measurement was performed using the electrode 2 obtained in Preparation Example 5. The measurement was performed with the measurement apparatus and the measurement conditions similar to those in Test Example 1 except that the following measurement solutions were used.

Measurement Solution: 1 M Potassium phosphate buffer (pH 8.0), 1 mM NADH, 10 mM glucose, 1 mg/mL NAD-dependent glucose dehydrogenase (manufactured by TOYOBO Co., Ltd., EC1. 1. 1. 47, 250 U/mg, hereinafter also referred to as GDH)

FIG. 6 shows the measurement result (solid line). The broken line shows the result of measurement under a condition without glucose and GDH, and the dotted line shows the result of measurement under a condition without NADH, glucose, and GDH.

In CV measurement 2, adding GDH and glucose resulted in an increase of the oxidation catalytic current and a decrease in the peak on the reduction side, and thus establishment of a conjugate system has been confirmed for the NAD/NADH regeneration system and the glucose oxidation reaction by GDH.

### Test Example 3: CV Measurement 3

Cyclic voltammetry (CV) measurement was performed using the electrode 2 obtained in Preparation Example 5.

The measurement was performed with the measurement apparatus and the measurement conditions similar to those in Test Example 1 except that the following measurement solutions were used.

Measurement Solution: 1 M potassium phosphate buffer (pH 8.0), 1 mM NAD⁺, 10 mM dihydroxyacetone (DHA), 1 mg/mL NAD-dependent glycerol dehydrogenase (manufactured by TOYOBO Co., Ltd., EC1. 1. 1. 6, 50 U/mg, hereinafter also referred to as GIDH)

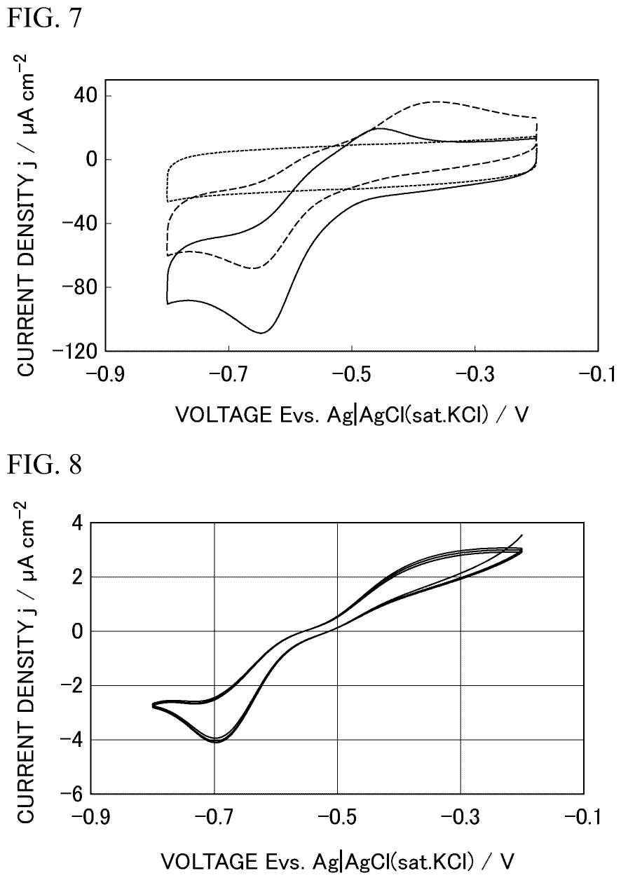

FIG. 7 shows the measurement result (solid line). The broken line shows the result of measurement under a condition without DHA and GIDH, and the dotted line shows the result of measurement under a condition without NAD*, DHA, and GIDH.

In CV measurement 3, adding GIDH and DHA resulted in an increase of the catalytic current on the reduction side and a decrease in the peak on the oxidation side, and thus establishment of a conjugate system has been confirmed for the NAD/NADH regeneration system and the DHA reduction reaction by GIDH.

### Preparation Example 6: Enzyme Electrode 3 Including Enzyme (a2) (rFoDH1β (Ec))

A glassy carbon electrode (GCE) was polished with alumina particles having outer diameters of 1.0 μm and 0.05 μm, and then ultrasonically washed with ultrapure water. Next, 10 mg of multi-walled carbon nanotubes (MWCNTs) were added to 10 mL of 1-methylpyrrolidone (NMP), then the suspension was sonicated for 2 hours, and 10 μL of the dispersion of the uniformly dispersed MWCNTs was added dropwise to the GCE and dried at 70° C. to obtain a CNT/GCE electrode. The CNT/GCE was immersed in an N,N-dimethylformamide (DMF) solution containing 10 mM 1-pyrenemethylamine hydrochloride (PyNH₂) and allowed to stand at room temperature for 1 hour to obtain a PyNH₂/CNT/GCE electrode. The prepared PyNH₂/CNT/GCE was washed with DMF and ultrapure water, and then 30 μL of a 1 mg/mL rFoDH1β (Ec) solution (dissolved in a 1 M potassium phosphate buffer (pH 8.0)) was added dropwise onto the electrode, the electrode was held under water vapor saturation conditions at 4° C. for 1 hour to adsorb the enzyme on the electrode surface, and thus an enzyme electrode 3 was obtained. The excess rFoDH1β (Ec) solution was removed, and then the enzyme electrode 3 was used for the next operation.

### Preparation Example 7: Enzyme Electrode 4 Including Enzyme (a2) (rFoDH1β (Ec)) and Enzyme (b) (GDH)

The enzyme electrode 3 was immersed in 1.5 mL of a 1 M potassium phosphate buffer (pH 8.0) containing 1 M glucose. To the buffer, 200 μL of a glucose dehydrogenase (GDH, GLD-311 manufactured by TOYOBO Co., Ltd.) solution (dissolved in 1 M potassium phosphate buffer (pH 8.0)) was added as a 10 mg/mL NAD(P)H or NAD(P) dependent oxidoreductase (b), NADH (dissolved in 1 M potassium phosphate buffer (pH 8.0)) was further added so that the final concentration was 10 mM, and the resulting mixture was adsorbed on the electrode surface of the enzyme electrode 3 to obtain an enzyme electrode 4 including the enzyme (a2), the enzyme (b), and NADH.

In the cyclic voltammetry (CV) measurement described below, the enzyme electrode 4 is to be used in a state of being immersed in the buffer. Therefore, the buffer contains the GDH in addition to the excess NADH. The excess NADH and GDH are absent in the vicinity of the electrode, that is, not immobilized and present in a portion to be regarded as a bulk solution. Meanwhile, the following CV and CA measurements are performed in a stationary solution, and therefore the excess NADH and GDH may be considered not to affect the electrochemical reaction.

### Preparation Example 8: Enzyme Electrode 5 Including Enzyme (a2) (rFoDH1β (Ec)) and Enzyme (b) (GDH)

The enzyme electrode 3 was immersed in 1.5 mL of a 1 M potassium phosphate buffer (pH 8.0) containing 50 mM NADH and 50 mM NAD⁺ to adsorb NADH and NAD⁺ on the electrode surface of the enzyme electrode 3. Next, to the buffer, 150 μL of a glucose dehydrogenase (GDH, GLD-311 manufactured by TOYOBO Co., Ltd.) solution (dissolved in 1 M potassium phosphate buffer (pH 8.0)) was added as a 10 mg/mL NAD(P)H or NAD(P) dependent oxidoreductase (b), and the resulting mixture was adsorbed on the electrode surface of the enzyme electrode 3 to obtain an enzyme electrode 5 including the enzyme (a2), the enzyme (b), and NADH. In the chronoamperometry (CA) measurement described below, the enzyme electrode 5 was used in a state of being immersed in the buffer. Glucose was added stepwise to the buffer in which the enzyme electrode 5 was immersed, and the value of the catalytic current generated by glucose oxidation was observed.

### Preparation Example 9 (Comparative Example): Enzyme Electrode 6 Containing Only Enzyme (b) (GDH)

A glassy carbon electrode (GCE) was polished with alumina particles having outer diameters of 1.0 μm and 0.05 μm, and then ultrasonically washed with ultrapure water. Next, 10 mg of multi-walled carbon nanotubes (MWCNTs) were added to 10 mL of 1-methylpyrrolidone (NMP), then the suspension was sonicated for 2 hours, and 10 μL of the dispersion of the uniformly dispersed MWCNTs was added dropwise to the GCE and dried at 70° C. to obtain a CNT/GCE electrode. The CNT/GCE was immersed in an N,N-dimethylformamide (DMF) solution containing 10 mM 1-pyrenemethylamine hydrochloride (PyNH₂) and allowed to stand at room temperature for 1 hour to obtain a PyNH₂/CNT/GCE electrode. The prepared PyNH₂/CNT/GCE was washed with DMF and ultrapure water, and then 30 μL of a solution containing GDH (0.4 mg/ml), NADH (10 mM), and glutaraldehyde (5%) (dissolved in a 1 M potassium phosphate buffer (pH 8.0)) was added dropwise onto the electrode, the electrode was allowed to stand at room temperature (1 h) to adsorb the enzyme on the electrode surface, and thus an enzyme electrode 6 was obtained.

### Preparation Example 10 (Comparative Example): Enzyme Electrode 7 Obtained by Adsorbing Enzyme (a2) (rFoDH1β (Ec)) on Enzyme Electrode 6

The enzyme electrode 6 was immersed in 1.5 mL of a 1 M potassium phosphate buffer (pH 8.0) containing 1 M glucose. To the buffer, 500 μL of a 1 mg/mL rFoDH1β (Ec) solution (dissolved in 1 M potassium phosphate buffer (pH 8.0)) was added and adsorbed on the electrode surface of the enzyme electrode 6 to obtain an enzyme electrode 7. In the cyclic voltammetry (CV) measurement described below, the enzyme electrode 7 is to be used in a state of being immersed in the buffer. Therefore, the buffer also contains the excess rFoDH1β (Ec). The excess rFoDH1β (Ec) is absent in the vicinity of the electrode, that is, not immobilized and present in a portion to be regarded as a bulk solution. Meanwhile, the following CV measurement is performed in a stationary solution, and therefore the excess rFoDH1β (Ec) may be considered not to affect the electrochemical reaction.

### Test Example 4: CV Measurement 4

Cyclic voltammetry (CV) measurement was performed using the enzyme electrode 3 obtained in Preparation Example 6.

The measurement apparatus and the measurement conditions were as follows.

Measurement apparatus: Electrochemical analyzer ALS660E (manufactured by BAS Inc.)
Solution: 1 M potassium phosphate buffer (pH 8.0), 50 mM NADH, 50 mM NAD⁺
Temperature: 25° C.
Atmosphere: Ar
Voltage sweep rate: 5 mVs⁻¹

FIG. 8 shows the measurement results.

In the enzyme electrode 3 in CV measurement 4, an oxidation wave and a reduction wave were observed, and a DET reaction was confirmed. In the enzyme electrode 3 using the rFoDH1β (Ec) expressed by E. coli, a larger catalytic current density was obtained than in the case of expression by an allogeneic strain. This is considered to be because the expression by E. coli caused an increase in the activity value of the rFoDH1β (Ec).

### Test Example 5: CV Measurement 5

Cyclic voltammetry (CV) measurement was performed using the enzyme electrode 4 obtained in Preparation Example 7. The measurement was performed with the measurement apparatus and the measurement conditions similar to those in Test Example 1 except that the following measurement solutions were used.

Measurement Solution: 1 M potassium phosphate buffer (pH 8.0), 10 mM NADH, 1 M glucose, 1 mg/mL NAD-dependent glucose dehydrogenase (GDH)

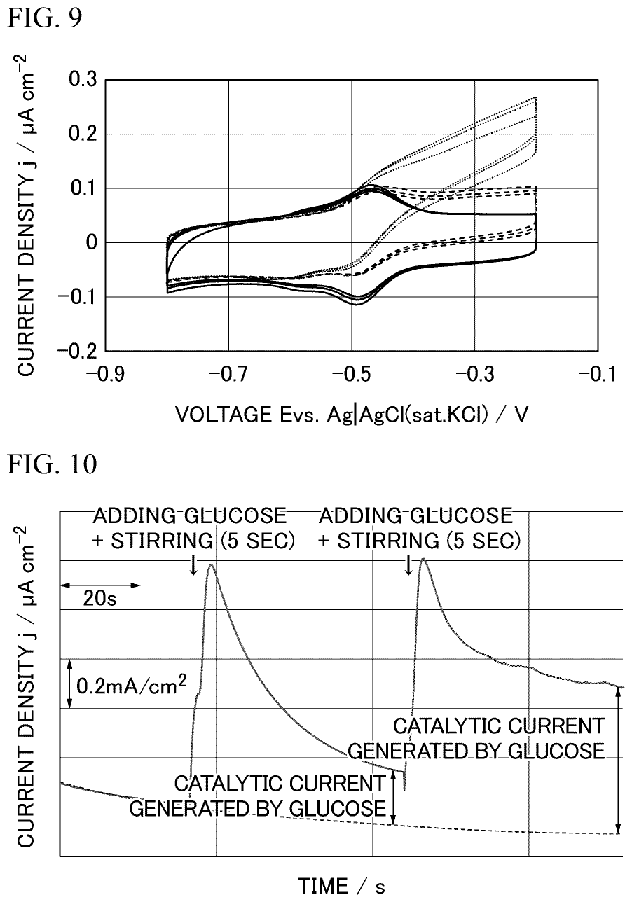

FIG. 9 shows the measurement result (solid line). The broken line shows the result of measurement under a condition without GDH, and the dotted line shows the result of measurement under a condition without NADH and GDH.

In CV measurement 5, adsorbing GDH and NADH on the electrode including rFoDH1β (Ec) resulted in an increase of the oxidation catalytic current, and thus establishment of a conjugate system has been confirmed for the NAD/NADH regeneration system by rFoDH1β (Ec) and the glucose oxidation reaction by GDH.

### Test Example 6: CA Measurement 6

Chronoamperometry (CA) measurement was performed using the enzyme electrode 5 obtained in Preparation Example 8.

The measurement was performed with the measurement apparatus and the measurement conditions similar to those in Test Example 4 except that the following measurement solutions were used.

Measurement Solution: 1 M potassium phosphate buffer (pH 8.0), 50 mM NADH, 1 mg/mL NAD-dependent glucose dehydrogenase

FIG. 10 shows the measurement result (solid line).

In CA measurement 6, the current value two minutes after the addition of GDH decreased to the extrapolation line in the case of adding no GDH. After the addition of glucose, the current value did not return to the extrapolation line. The increase in the current value depended on the amount of glucose added, so that it is clear that the current is associated with the oxidation of glucose.

### Test Example 7 (Comparative Example): CV Measurement 7

Cyclic voltammetry (CV) measurement was performed using the enzyme electrode 6 obtained in Preparation Example 9 and the enzyme electrode 7 obtained in Preparation Example 10. The measurement was performed with the measurement apparatus and the measurement conditions similar to those in Test Example 4 except that the following measurement solutions were used.

Measurement Solution: 1 M potassium phosphate buffer (pH 8.0), 10 mM NADH, 1 M glucose, 0.25 mg/mL rFoDH1β

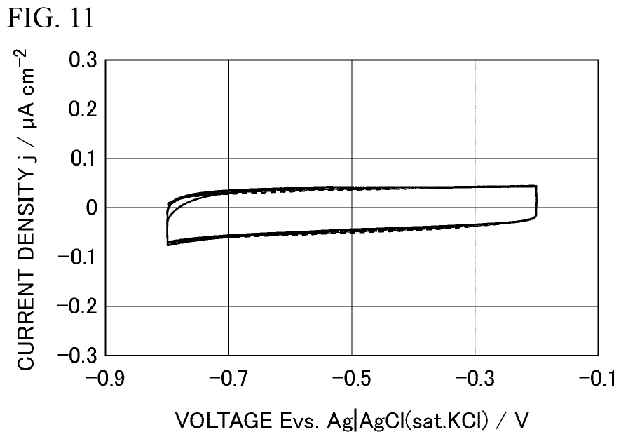

FIG. 11 shows the measurement results. The broken line shows the result of the enzyme electrode 6, and the solid line shows the result of the enzyme electrode 7. In both the enzyme electrodes 6 and 7, an increase in the catalytic current was not observed. The reason why an increase in the catalytic current was not observed in the enzyme electrode 6 is that the enzyme electrode 6 included only GDH and did not include rFoDH1β (Ec).

The reason of the result of the enzyme electrode 7 is that although the enzyme electrode 7 included rFoDH1β (Ec) and GDH, the first catalyst layer including rFoDH1β (Ec) was layered on the second catalyst layer including GDH on the electrode base material. From the above, it can be clearly said that the technically significant fact is that the enzyme electrode of the present disclosure includes the first catalyst layer and the second catalyst layer that are layered in this order on the electrode base material.

### REFERENCE SIGNS LIST

1: enzyme electrode
2: enzyme (a1)
3: enzyme (b)
4: NAD(P)
5: NAD(P)H
6: electrode base material
7: substrate
8: first catalyst layer
9: second catalyst layer

1. An enzyme electrode comprising:
an electrode base material;
a first catalyst layer on the electrode base material, the first catalyst layer including an enzyme (a1) capable of catalyzing an oxidation-reduction reaction of a redox couple of NAD(P)H and NAD(P), the enzyme (a1) capable of direct electron exchange between the enzyme (a1) and the electrode base material accompanying the oxidation-reduction reaction; and
a second catalyst layer on the first catalyst layer, the second catalyst layer including an NAD(P)H or NAD(P) dependent oxidoreductase (b) and NAD(P)H and/or NAD(P).

2. The enzyme electrode according to claim 1, wherein the enzyme (a1) has one flavin mononucleotide and at least one Fe—S cluster, the one flavin mononucleotide and the at least one Fe—S cluster have a distance of 2 nm or less from each other, and the at least one Fe—S cluster is present at a distance of 2 nm or less from a surface of the enzyme (a1).

3. The enzyme electrode according to claim 2, wherein the Fe—S cluster is a 2Fe-2S cluster.

4. The enzyme electrode according to claim 2, wherein the distance is 1.5 nm or less.

5. The enzyme electrode according to claim 2, wherein the distance is 1 nm or less.

6. The enzyme electrode according to claim 1, wherein the enzyme (a1) has one flavin mononucleotide and a plurality of Fe—S clusters, the one flavin mononucleotide and at least a first of the plurality of Fe—S clusters have a distance of 2 nm or less from each other, and at least a second of the plurality of Fe—S clusters is present at a distance of 2 nm or less from a surface of the enzyme (a1).

7. The enzyme electrode according to claim 6, wherein the one flavin mononucleotide has the distance of 2 nm or less from each of the first and the second of the plurality of Fe—S clusters.

8. The enzyme electrode according to claim 6, wherein the first and the second of the plurality of Fe—S clusters have a distance of 3 nm or less from each other.

9. The enzyme electrode according to claim 6, wherein each of the first and the second of the plurality of Fe—S clusters has a distance of 2 nm or less from the surface of the enzyme (a1).

10. The enzyme electrode according to claim 1, wherein the enzyme (a1) contains a β subunit of a formate dehydrogenase derived from a methanol-assimilating bacterium.

11. The enzyme electrode according to claim 1, wherein an amount of the enzyme (a1) in the first catalyst layer is 0.05 pmol/cm² to 1000 pmol/cm² with respect to a surface area of the electrode base material.

12. The enzyme electrode according to claim 1,
wherein a surface of the electrode base material is covered with a surface-modifying group, and
the surface-modifying group has a charge opposite to a surface charge of a portion on the surface of the enzyme (a1), the portion located at a distance of 2 nm or less from the one or more Fe—S clusters in the enzyme (a1).

13. The enzyme electrode according to claim 1, wherein a material of the electrode base material is at least one selected from a carbon particle, a metal, and a conductive polymer,
wherein the carbon particle is at least one selected from activated carbon, carbon black, and carbon nanotubes,
the metal is at least one selected from gold, platinum, copper, palladium, and titanium, and
the conductive polymer is at least one selected from polyaniline and polypyrrole.

14. The enzyme electrode according to claim 1, wherein a substrate of the NAD(P)H or NAD(P) dependent oxidoreductase (b) is glycerol.

15. A biosensor comprising the enzyme electrode according to claim 1.

16. A bioreactor comprising the enzyme electrode according to claim 1.

17. A biofuel cell comprising the enzyme electrode according to claim 1.

18. An enzyme capable of catalyzing an oxidation-reduction reaction of a redox couple of NAD(P)H and NAD(P),
the enzyme comprising one flavin mononucleotide and two Fe—S clusters,
wherein the one flavin mononucleotide and at least one of the two Fe—S clusters have a distance of 2 nm or less, and at least one of the two Fe—S clusters is present at a distance of 2 nm or less from a surface of the enzyme.

19. The enzyme according to claim 18, capable of direct electron exchange between the enzyme and an electrode base material when used in an electrode, the direct electron exchange accompanying the oxidation-reduction reaction.

20. The enzyme according to claim 18, having an average molecular weight of 50000 to 100000.


SEQUENCE LISTING (2 sequences)

SEQ ID NO: 1 - protein, 989 aa, Methylorubrum extorquens
MSNGPEPHGNKIEQPEIRADERQDAGGPANGAPSTSGGAYSQGAKSGGQAAPDPSGSYGIKDAPVAPATIAFEFDGQQVEAQPGETIWAVAKRLGTHIPHLCHKPDPGYRPDGNCRACMVEIEGERVLAASCKRTPAIGMKVKSATERATKARAMVLELLVADQPERATSHDPSSHFWVQADVLDVTESRFPAAERWTSDVSHPAMSVNLDACIQCNLCVRACREVQVNDVIGMAYRAAGSKVVFDFDDPMGGSTCVACGECVQACPTGALMPAAYLDANQTRTVYPDREVKSLCPYCGVGCQVSYKVKDERIVYAEGVNGPANQNRLCVKGRFGFDYVHHPHRLTVPLIRLENVPKDANDQVDPANPWTHFREATWEEALDRAAGGLKAIRDTNGRKALAGFGSAKGSNEEAYLFQKLVRLGFGTNNVDHCTRLCHASSVAALMEGLNSGAVTAPFSAALDAEVIVVIGANPTVNHPVAATFLKNAVKQRGAKLIIMDPRRQTLSRHAYRHLAFRPGSDVAMLNAMLNVIVTEGLYDEQYIAGYTENFEALREKIVDFTPEKMASVCGIDAETLREVARLYARAKSSLIFWGMGVSQHVHGTDNSRCLIALALITGQIGRPGTGLHPLRGQNNVQGASDAGLIPMVYPDYQSVEKDAVRELFEEFWGQSLDPQKGLTVVEIMRAIHAGEIRGMFVEGENPAMSDPDLNHARHALAMLDHLVVQDLFLTETAFHADVVLPASAFAEKAGTFTNTDRRVQIAQPVVAPPGDARQDWWIIQELARRLDLDWNYGGPADIFAEMAQVMPSLNNITWERLEREGAVTYPVDAPDQPGNEIIFYAGFPTESGRAKIVPAAIVPPDEVPDDEFPMVLSTGRVLEHWHTGSMTRRAGVLDALEPEAVAFMAPKELYRLGLRPGGSMRLETRRGAVVLKVRSDRDVPIGMIFMPFCYAEAAANLLTNPALDPLGKIPEFKFCAARVVPAEAAPMAAE

SEQ ID NO: 2 - protein, 572 aa, Methylorubrum extorquens
MSEASGTVRSFAHPGRGRNVARAVPKGRQVDPHAKVEIEELLGTRPRQRDLLIEHLHLIQDTYGQISADHLAALADEMSLAFAEVFETATFYAHFDVVKEGEADIPRLTIRVCDSITCAMFGADELLETLQRELASDAVRVVRAPCVGLCDHAPAVEVGHNFLHRADLASVRAAVEAEDTHAHIPTYVDYDAYRAGGGYATLERLRSGELPVDDVLKVLDDGGLRGLGGAGFPTGRKWRSVRGEPGPRLMAVNGDEGEPGTFKDQLYLNTDPHRFLEGMLIGAHVVEAADVYIYLRDEYPISREILAREIAKLPEGGTRIHLRRGAGAYICGEESSLIESLEGKRGLPRHKPPFPFQVGLFNRPTLINNIETLFWVRDLIERGAEWWKSHGRNGRVGLRSYSVSGRVKEPGVKLAPAGLTIQELIDEYCGGISDGHSFAAYLPGGASGGILPASMNDIPLDFGTLEKYGCFIGSAAVVILSDQDDVRGAALNLMKFFEDESCGQCTPCRSGTQKARMLMENGVWDTDLLGELAQCMRDASICGLGQAASNPVSTVIKYFPDLFPEPRAVAAE